(19) United States
(12) Patent Application Publication
(10) Pub. No.: US 2006/0240452 A1
(43) Pub. Date: Oct. 26, 2006

(54) Flourecent quinacridone derivatives

(75) Inventors: Zbigniev Skrzypczynski, Verona, WI (US); Robert Roeven, Stoughton, WI (US)

Correspondence Address: Medlen & Carroll, LLP, Suite 350, 101 Howard Street, San Francisco, CA, 94105, US

(73) Assignee: Third Wave Technologies, Inc.

(21) Appl. No.: 11/352,612

(22) Filed: Feb. 13, 2006

Related U.S. Application Data
(60) Provisional application No. 60/652,268, filed on Feb. 11, 2005.

(51) Int. Cl. C12Q 1/68; C07H 21/02; C07D 471/02

(52) U.S. Cl. 435/6; 536/25.32; 546/49; 546/21

(57) ABSTRACT

The present invention relates to methods and compositions utilizing fluorescent quinacridone derivatives. In particular, the present invention relates to the use of fluorescent quinacridone derivatives for the labeling and detection of riucleic acids. The present invention thus provides improved compositions and methods for labeling biological molecules useful in the detection of nucleic acids and other biological molecules.

This application claims priority to provisional patent application Ser. No. 60/652,268, filed Feb. 11, 2005, which is herein incorporated by reference in its entirety.

### FIELD OF THE INVENTION

The present invention relates to methods and compositions utilizing fluorescent quinacridone derivatives. In particular, the present invention relates to the use of fluorescent quinacridone derivatives for the labeling and detection of biological molecules, including nucleic acid molecules.

### BACKGROUND OF THE INVENTION

Traditional methods for detecting biological compounds in vivo and in vitro rely on the use of radioactive markers. For example, these methods commonly use radiolabeled probes such as nucleic acids labeled with ³²p or ³⁵S and proteins labeled with ³⁵S or ¹²⁵I to detect biological molecules. These labels are effective because of the high degree of sensitivity for the detection of radioactivity. However, many basic difficulties exist with the use of radioisotopes. Such problems include the need for specially trained personnel, general safety issues when working with radioactivity, inherently short half-lives with many commonly used isotopes, and disposal problems due to full landfills and governmental regulations. As a result, current efforts have shifted to utilizing non-radioactive methods of detecting biological compounds. These methods often consist of the use of fluorescent molecules as tags or the use of chemiluminescence as a method of detection.

While a variety of fluorescent labels are available, a need still exists in the art for fluorescent compounds that have one or more desired properties, including stability, both chemically and to light, high quantum efficiency, and are relatively insensitive to interactions with a variety of molecules, as well as variations in medium, have high light absorption and emission characteristics, are relatively insensitive to self-quenching, and are able to be readily attached to a wide variety of molecules under varying conditions without adversely affecting the fluorescent characteristics.

### SUMMARY OF THE INVENTION

The present invention relates to methods and compositions utilizing fluorescent quinacridone derivatives. In particular, the present invention relates to the use of fluorescent quinacridone derivatives for the labeling and detection of biological molecules, including nucleic acids.

The present invention provides a composition comprising a biological molecule, wherein the biological molecule comprises a quinacridone. The present invention is not limited to a particular biological molecule. A variety of biological molecules are suitable for use in the compositions and methods of the present invention including, but not limited to, proteins (e.g., antibodies, polypeptides, and peptides), carbohydrates, lipids, and nucleic acids. In some preferred embodiments, the biological molecule is a nucleic acid. In some embodiments, the quinacridone has the structure:

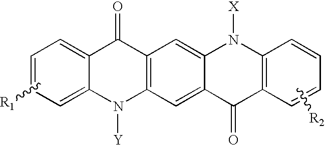

In some embodiments, X and/or Y are chemical groups that increase solubility of the quinacridone. In some embodiments, X and/or Y comprise a polar group (e.g., an alcohol group). In some embodiments, X is (CH₂)₆—O—(CH₂)₃—OH and Y is (CH₂)₆—O—(CH₂)₃—OH. In other embodiments, X and Y are independently hydrogen, halogen, amide, hydroxyl, cyano, nitro, azido mono- or di-nitro-substituted benzyl, amino, mono- or di-C₁-C₄ alkyl-substituted amino, sulphydryl, carbonyl, carboxyl, C₁-C₆ alkoxy, acrylate, vinyl, styryl, aryl, heteroaryl, C₁-C₂₀ alkyl, aralkyl, sulphonate, sulphonic acid, quaternary ammonium, E-F or (CH₂)ₙ-G, wherein E is a spacer group having a chain from 1-60 atoms selected from the group consisting of carbon, nitrogen, oxygen, sulphur and phosphorus atoms and F is a target bonding group, and G is selected from the group consisting of sulphonate, sulphate, phosphonates, phosphate, quaternary ammonium and carboxyl and n is an integer from 1 to 6. In some embodiments, R₁ and R₂ are independently hydrogen, halogen, amide, hydroxyl, cyano, nitro, mono- or di-nitro- substituted benzyl, amino, mono- or di-C₁-C₄ alkyl-substituted amino, sulphydryl, carbonyl, carboxyl, C₁-C₆ alkoxy, acrylate, vinyl, styryl, aryl, heteroaryl, C₁-C₂₀ alkyl, aralkyl, sulphonate, sulphonic acid, quaternary ammonium, E-F and (CH₂)ₙ-G, wherein E is a spacer group having a chain from 1-60 atoms selected from the group consisting of carbon, nitrogen, oxygen, sulphur and phosphorus atoms and F is a target bonding group, and G is selected from the group consisting of sulphonate, sulphate, phosphonates, phosphate, quaternary ammonium and carboxyl and n is an integer from 1 to 6. The present invention is not limited to a particular nucleic acid. A variety of nucleic acids are contemplated, including, but not limited to, ssDNA, ssRNA, dsDNA, dsRNA, and PNA. In some embodiments, the quinacridone is covalently linked to the nucleic acid. In some embodiments, the nucleic acid is an oligonucleotide. In some embodiments, the oligonucleotide further comprises a fluorescence quenching molecule.

The present invention further provides a composition comprising a phosphoramidite, wherein the phosphoramidite comprises a quinacridone. In some embodiments, the quinacridone has the structure: 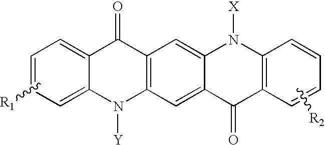
In some embodiments, X and/or Y are chemical groups that increase solubility of the quinacridone. In some embodiments, X and/or Y comprise a polar group (e.g., an alcohol group). In some embodiments, X is (CH₂)₆—O—(CH₂)₃—OH and Y is (CH₂)₆—O—(CH₂)₃—OH. In other embodiments, X and Y are independently hydrogen, halogen, amide, hydroxyl, cyano, nitro, azido, mono- or di-nitro-substituted benzyl, amino, mono- or di-C₁-C₄ alkyl-substituted amino, sulphydryl, carbonyl, carboxyl, C₁-C₆ alkoxy, acrylate, vinyl, styryl, aryl, heteroaryl, C₁-C₂₀ alkyl, aralkyl, sulphonate, sulphonic acid, quaternary ammonium, E-F or (CH₂)ₙ-G, wherein E is a spacer group having a chain from 1-60 atoms selected from the group consisting of carbon, nitrogen, oxygen, sulphur and phosphorus atoms and F is a target bonding group, and G is selected from the group consisting of sulphonate, sulphate, phosphonates, phosphate, quaternary ammonium and carboxyl and n is an integer from 1 to 6. In some embodiments, R₁ and R₂ are independently hydrogen, halogen, amide, hydroxyl, cyano, nitro, azido, mono- or di-nitro-substituted benzyl, amino, mono- or di-C₁-C₄ alkyl-substituted amino, sulphydryl, carbonyl, carboxyl, C₁-C₆ alkoxy, acrylate, vinyl, styryl, aryl, heteroaryl, C₁-C₂₀ alkyl, aralkyl, sulphonate, sulphonic acid, quaternary ammonium, E-F and (CH₂)ₙ-G, wherein E is a spacer group having a chain from 1-60 atoms selected from the group consisting of carbon, nitrogen, oxygen, sulphur and phosphorus atoms and F is a target bonding group, and G is selected from the group consisting of sulphonate, sulphate, phosphonates, phosphate, quaternary ammonium and carboxyl and n is an integer from 1 to 6.

The present invention also provides a kit comprising a biological molecule, wherein the biological molecule comprises a quinacridone. In some embodiments, the quinacridone has the structure: 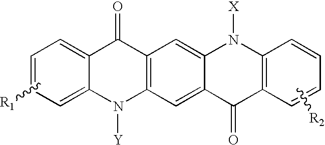
In some embodiments, X is (CH₂)₆—O—(CH₂)₃—OH and Y is (CH₂)₆—O—(CH₂)₃—OH. In other embodiments, X and Y are independently hydrogen, halogen, amide, hydroxyl, cyano, nitro, azido, mono- or di-nitro-substituted benzyl, amino, mono- or di-C₁-C₄ alkyl-substituted amino, sulphydryl, carbonyl, carboxyl, C₁-C₆ alkoxy, acrylate, vinyl, styryl, aryl, heteroaryl, C₁-C₂₀ alkyl, aralkyl, sulphonate, sulphonic acid, quaternary ammonium, E-F or (CH₂)ₙ-G, wherein E is a spacer group having a chain from 1-60 atoms selected from the group consisting of carbon, nitrogen, oxygen, sulphur and phosphorus atoms and F is a target bonding group, and G is selected from the group consisting of sulphonate, sulphate, phosphonates, phosphate, quaternary ammonium and carboxyl and n is an integer from 1 to 6. In some embodiments, R₁ and R₂ are independently hydrogen, halogen, amide, hydroxyl, cyano, nitro, azido, mono- or di-nitro-substituted benzyl, amino, mono- or di-C₁-C₄ alkyl-substituted amino, sulphydryl, carbonyl, carboxyl, C₁-C₆ alkoxy, acrylate, vinyl, styryl, aryl, heteroaryl, C₁-C₂₀ alkyl, aralkyl, sulphonate, sulphonic acid, quaternary ammonium, E-F and (CH₂)ₙ-G, wherein E is a spacer group having a chain from 1-60 atoms selected from the group consisting of carbon, nitrogen, oxygen, sulphur and phosphorus atoms and F is a target bonding group, and G is selected from the group consisting of sulphonate, sulphate, phosphonates, phosphate, quaternary ammonium and carboxyl and n is an integer from 1 to 6. The present invention is not limited to a particular biological molecule. A variety of biological molecules are suitable for use in the compositions and methods of the present invention including, but not limited to, proteins (e.g., antibodies, polypeptides, and peptides), carbohydrates, lipids, and nucleic acids. In some preferred embodiments, the biological molecule is a nucleic acid. A variety of nucleic acids are contemplated, including, but not limited to, ssDNA, ssRNA, dsDNA, dsRNA, and PNA. In some embodiments, the quinacridone is covalently linked to the nucleic acid. In some embodiments, the nucleic acid is an oligonucleotide. In some embodiments, the oligonucleotide further comprises a fluorescence quenching molecule. In some embodiments, the kit further comprises a second nucleic acid, wherein the second nucleic acid comprises a fluorescent molecule with a different fluorescence emission spectrum than the quinacridone. In some embodiments, the fluorescent molecule is a second quinacridone. In some embodiments, the kit further comprises reagents for performing a detection assay including, but not limited to, the INVADER assay, the TAQMAN assay, the SNP-IT assay, a Southern blot, and an array assay. In other embodiments, the kit further comprises reagents for performing a nucleic acid sequencing assay.

The present invention additionally provides a method of detecting a target nucleic acid sequence, comprising, providing a nucleic acid comprising a quinacridone; a sample comprising target nucleic acid; and contacting the sample with the nucleic acid under conditions such that the target nucleic acid sequence is detected. In some embodiments, the quinacridone has the structure: 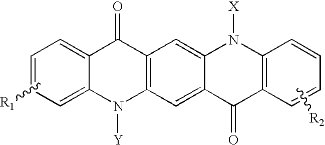
In some embodiments, X and/or Y are chemical groups that increase solubility of the quinacridone. In some embodiments, X and/or Y comprise a polar group (e.g., an alcohol group). In some embodiments, X is (CH₂)₆—O—(CH₂)₃—OH and Y is (CH₂)₆—O—(CH₂)₃—OH. In other embodiments, X and Y are independently hydrogen, halogen, amide, hydroxyl, cyano, nitro, azido, mono- or di-nitro-substituted benzyl, amino, mono- or di-C₁-C₄ alkyl-substituted amino, sulphydryl, carbonyl, carboxyl, C₁-C₆ alkoxy, acrylate, vinyl, styryl, aryl, heteroaryl, C₁-C₂₀ alkyl, aralkyl, sulphonate, sulphonic acid, quaternary ammonium, E-F or (CH₂)ₙ-G, wherein E is a spacer group having a chain from 1-60 atoms selected from the group consisting of carbon, nitrogen, oxygen, sulphur and phosphorus atoms and F is a target bonding group, and G is selected from the group consisting of sulphonate, sulphate, phosphonates, phosphate, quaternary ammonium and carboxyl and n is an integer from 1 to 6. In some embodiments, R₁ and R₂ are independently hydrogen, halogen, amide, hydroxyl, cyano, nitro, azido mono- or di-nitro-substituted benzyl, amino, mono- or di-C₁-C₄ alkyl-substituted amino, sulphydryl, carbonyl, carboxyl, C₁-C₆ alkoxy, acrylate, vinyl, styryl, aryl, heteroaryl, C₁-C₂₀ alkyl, aralkyl, sulphonate, sulphonic acid, quaternary ammonium, E-F and (CH₂)ₙ-G, wherein E is a spacer group having a chain from 1-60 atoms selected from the group consisting of carbon, nitrogen, oxygen, sulphur and phosphorus atoms and F is a target bonding group, and G is selected from the group consisting of sulphonate, sulphate, phosphonates, phosphate, quaternary ammonium and carboxyl and n is an integer from 1 to 6. In some embodiments, the contacting step comprises a nucleic acid detection assay. In some embodiments, the detection assay includes, but is not limited to, the INVADER assay, the TAQMAN assay, the SNP-IT assay, a Southern blot, and an array assay.

### DESCRIPTION OF THE FIGURES

FIG. 1 shows the fluorescence emission spectrum for an illustrative fluorescent quinacridone derivative of the present invention.

FIG. 2 shows the fluorescence emission spectrum for an illustrative fluorescent quinacridone derivative of the present invention.

FIG. 3 shows the fluorescence emission spectrum for an illustrative fluorescent quinacridone derivative of the present invention.

FIG. 4 shows absorbance and emission spectra for an oligonucleotide labeled with an illustrative fluorescent quinacridone derivative of the present invention.

FIGS. 5 A-C show the results of monoplex INVADER assays using ZB2, FAM, and Red dye labeled oligonucleotide.

FIG. 6 shows the results of triplex INVADER assays using ZB2, FAM, and Red dye labeled oligonucleotide.

FIG. 7 shows monoplex reactions of the PSS assay, using FAM and ZB2 reporter dyes.

FIG. 8 shows biplex reactions of ZB2- and RED dye with FAM.

FIG. 9 shows the results of reactions using ZB2 FRET oligos.

FIG. 10 illustrates a comparison between 1273-74 and the FAM and RED FRET oligos.

### DEFINITIONS

As used herein, the term “fluorescent quinacridone” refers to any quinacridone derivative that, when excited, emits light of a different wavelength than the excitation wavelength.

As used herein, the term “fluorescence quenching molecule” refers to a molecule that absorbs energy transferred from a particular fluorophore (e.g., the fluorescent quinacridone derivatives of the present invention). In order for energy transfer to occur, the emission spectrum of the fluorophore and the absorption spectrum of the quencher should overlap.

As used herein, the terms “X, Y, R₁ and R₂” refer to any atom or molecule attached to a molecule (e.g., a quinacridone of the present invention).

As used herein, the term “nucleic acid molecule” refers to any nucleic acid containing molecule, including but not limited to, DNA (single and double-stranded), RNA (single and double-stranded), and protein nucleic acid (PNA). The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyamino-methyl-2-thiouracil, beta-D-mannosylqueosine, 5-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids′ bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc).

As used herein, the term “sample template” refers to nucleic acid originating from a sample that is analyzed for the presence of “target.” In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (e.g., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer should be sufficiently long to prime the synthesis of extension products in the presence of the inducing agent. The exact lengths of the primers will depend on many factors, including temperature, source of primer and the use of the method.

As used herein, the term “probe” refers to an oligonucleotide, whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that probes used in the present invention can be labeled with a “reporter molecule,” so that they are detectable in a detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target” refers to the region of nucleic acid that is sought to be sorted out from other nucleic acid sequences. A “probe” is sometimes, but not always, designed to be complementary to the “target.” In some embodiments, the target nucleic acid is a region containing a mutation or polymorphism of interest.

As used herein, the term “polymerase chain reaction” (“PCR”) refers to the method of K. B. Mullis U.S. Pat. Nos. 4,683,195 4,683,202, and 4,965,188, hereby incorporated by reference, which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”.

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “amplification reagents” refers to those reagents (deoxyribonucleotide triphosphates, buffer, etc.), needed for amplification except for primers, nucleic acid template and the amplification enzyme. Typically, amplification reagents along with other reaction components are placed and contained in a reaction vessel (test tube, microwell, etc.).

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe (e.g., labeled with a quinacridone derivative of the present invention) to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 [1989]).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe (e.g., labeled with a quinacridone derivative of the present invention) to detect RNA species complementary to the probe used. Northern blots are a standard tool of molecular biologists (J. Sambrook, et al., supra, pp 7.39-7.52 [1989]).

### DETAILED DESCRIPTION OF THE INVENTION

The present invention relates to.methods and compositions utilizing fluorescent quinacridone derivatives. The present invention relates to the use of fluorescent quinacridone derivatives for the labeling and detection of biological molecules. In particular, the present invention provides nucleic acids labeled with fluorescent quinacridone derivatives and diagnostic methods utilizing such nucleic acids.

I. Quinacridones

Currently, quinacridones are produced on a large scale by many manufacturers and are used as ingredients of paints and as color additives in the polymer and textile industries (for commercial sources, See e.g., Lona, Dadar, Mumbai, India; Bayer, Pittsburgh, Pa.; Amantech, Raleigh, N.C.; Keystone Aniline Corp., Chicago, Ill.; and SunChemicals Cincinatti, Ohio). Quinacridones are also used in the preparation of fluorescent materials, inks for printing devices, and preparation of Light Emitting Diodes (e.g H.W. Sands Corp, Jupiter, Fla.). The spectral properties of quinacridones have been studied (See e.g., Klien et al., Chem. Commun., 561-562 [2001]; McDonald et al., J. Am. Chem. Soc., 122:4972 [2000]; U.S. Pat. Nos. 5,561,232; 5,725,651; 6,013,777; 6,127,549; 5,886,160; and 6,031,100; each of which is herein incorporated by reference).

Quinacridone pigments/dyes can be synthesized as linear-trans-quinacridones, linear-cis-quinacridones, and non-linear-quinacridones (For a review See e.g., Chemical Review 67:1 [1967]). Basic structures are shown below:

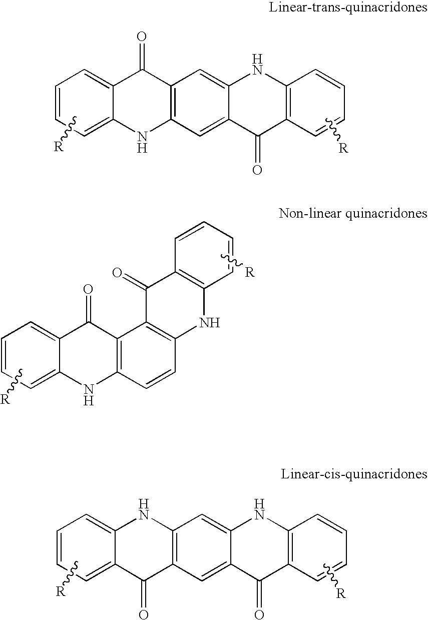

Many of the known quinacridone derivatives are highly insoluble and can have polymorphic crystalline structures. While the insolubility is desired in the dye and pigment industry, it is frequently not desired for other applications (e.g., labeling of biological molecules).

In some embodiments, the present invention provides quinacridones modified to increase their solubility and/or fluorescence (e.g., alter fluorescence emission spectra) for use in the labeling of biological molecules. The present invention is not limited to a particular quinacridone derivative (e.g., linear or non-linear). Any derivative having the desired properties (e.g., solubility and fluorescence) may be utilized in the methods and compositions of the present invention. In some embodiments, quinacridone derivatives are modified by alkylation of the secondary nitrogen atoms of the quinacridone molecule of interest. In preferred embodiments, this procedure converts insoluble quinacridone pigments into fluorescent derivatives that show increased solubility in organic solvents. Example 2 below describes one preferred method of modifying quinacridone derivatives to increase their solubility and fluorescence. In other embodiments, quinacridones are synthesized ab initio using starting materials containing desired structural properties or moieties.

In some embodiments, the following derivative is utilized: 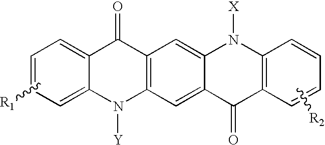
In some embodiments, X and Y are each (CH₂)₆—O—(CH₂)₃—OH, resulting in the following quinacridone: 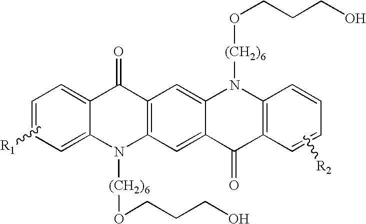
The present invention is not limited to particular X and Y groups. For example, in some embodiments, X and Y are organic moieties containing functional groups suitable for conjugation (e.g., including, but not limited to, amino, carboxyl, aldehyde, sulfhydryl, phosphate, thiophosphate, and dithiophosphate). In other embodiments, X is (CH₂)6-O—(CH₂)3-Z, where Z=a protected or unprotected reactive or functional group. In other embodiments, X and Y are independently hydrogen, halogen, amide, hydroxyl, cyano, nitro, azido, mono- or di-nitro-substituted benzyl, amino, mono- or di-C₁-C₄ alkyl-substituted amino, sulphydryl, carbonyl, carboxyl, C₁-C₆ alkoxy, acrylate, vinyl, styryl, aryl, heteroaryl, C₁-C₂₀ alkyl, aralkyl, sulphonate, sulphonic acid, quaternary ammonium, E-F or (CH₂)ₙ-G, where E is a spacer group having a chain from 1-60 atoms selected from the group consisting of carbon, nitrogen, oxygen, sulphur and phosphorus atoms and F is a target bonding group, and G is sulphonate, sulphate, phosphonates, phosphate, quaternary ammonium and carboxyl and n is an integer from 1 to 6.

The present invention is not limited to the linear-trans quinacridone derivative described herein. It is contemplated that linear linear-cis and non-linear derivatives can also be converted into the desired soluble and fluorescent derivatives. Candidate quinacridones can be screened for solubility and fluorescence using techniques well known in the art (e.g., those described in the examples below).

The present invention is also not limited to particular R₁ and R₂ groups. Any suitable substituent groups may be utilized, including but not limited to, hydrogen, halogen, amide, hydroxyl, cyano, nitro, azido, mono- or di-nitro-substituted benzyl, amino, mono- or di-C₁-C₄ alkyl-substituted amino, sulphydryl, carbonyl, carboxyl, C₁-C₆ alkoxy, acrylate, vinyl, styryl, aryl, heteroaryl, C₁-C₂₀ alkyl, aralkyl, sulphonate, sulphonic acid, quaternary ammonium, E-F or (CH₂)ₙ-G, where E is a spacer group having a chain from 1-60 atoms selected from the group consisting of carbon, nitrogen, oxygen, sulphur and phosphorus atoms and F is a target bonding group, and G is sulphonate, sulphate, phosphonates, phosphate, quaternary ammonium and carboxyl and n is an integer from 1 to 6.

Suitable spacer groups E may contain 1-60 chain atoms selected from the group consisting of carbon, nitrogen, oxygen, sulphur and phosphorus. For example the spacer group may be: —(CHR′)ₚ— —{(CHR′)_{q}—O—(CHR′)ᵣ}ₛ—{(CHR′)_{q}—NR′—(CHR′)ᵣ}ₛ—]—}(CHR′)_{q}—(CH═CH)—(CHR′)ᵣ}ₛ— —{(CHR′)_{q}—Ar—(CHR′)ᵣ}ₛ— —{(CHR′)_{q}—CO—NR′—(CHR′)ᵣ}ₛ— —{(CHR′)_{q}—CO—Ar—NR′—(CHR′)ᵣ}ₛ— where R′ is hydrogen, C₁-C₄ alkyl or aryl, which may be optionally substituted with sulphonate, Ar is phenylene, optionally substituted with sulphonate, p is 1-20, preferably 1-10, q is 0-10, r is 1-10 and s is 1-5. In some embodiments, the target bonding group F is a reactive or functional group. A reactive group of a dye of formula (I) can react under suitable conditions with a functional group of a target material; a functional group of a dye of formula (I) can react under suitable conditions with a reactive group of the target material such that the target material becomes labelled with the compound. Preferably, when F is a reactive group, it is selected from succinimidyl ester, sulpho-succinimidyl ester, isothiocyanate, maleimide, haloacetamide, acid halide, vinylsulphone, dichlorotriazine, carbodiimide, hydrazide or phosphoramidite. Preferably, when F is a functional group, it is selected from hydroxy, amino, sulphydryl, imidazole, carbonyl including aldehyde and ketone, phosphate or thiophosphate.

R groups can be attached at any of the available positions. Preferred R groups are those that result in a quinacridone with more desired spectral properties (e.g., excitation/emission wavelengths). Chemical Review, 1967, 67(1), 1-18 describes a variety of substituted quinacridones having different spectral properties. The present invention further contemplates thioquinacridones of similar structure to the quinacridones disclosed above in order to alter the fluorescent or other properties of the label. Thioquinacridones replace with sulphur atoms the central oxygen atoms located para- to the central nitrogen atoms. A generic thioquinacridone structure is shown below: 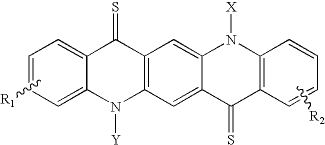
II. Quinacridone Nucleic Acids

In some embodiments, the present invention provides quinacridone-nucleic acid conjugates. Such conjugates find use in a variety of diagnostic and analytical methods.

A. Conjugation of Fluorescent Quinacridone Dyes to Nucleic Acids

In some embodiments, the present invention provides nucleic acids labeled with fluorescent quinacridone derivatives. The present invention is not limited to a particular quinacridone or nucleic acid. The present invention is also not limited to a particular method of synthesizing quinacridones and conjugating quinacridones to nucleic acids. The below description and examples provide exemplary non-limiting methods.

The present invention is not limited to the use of a particular nucleic acid for conjugation. Any nucleic acid may be utilized, including but not limited to, ssDNA, dsDNA, ssRNA, MRNA, tRNA, dsRNA, and PNA. The present invention is also not limited to a particular length of nucleic acid molecule.

In some preferred embodiments, oligonucleotides are utilized for labeling. In some embodiments, oligonucleotides are labeled at the 5′ end. In other embodiments, oligonucleotides are labeled at the 3′ end. In yet other embodiments, oligonucleotides are labeled internally. In some embodiments, nucleic acids are labeled in one location (e.g., 3′, 5′, or internally) with a fluorescent quinacridone dye. In other embodiments, nucleic acids contain greater than one label. In some embodiments, the labels are the same fluorescent quinacridone derivative. In other embodiments, the labels comprise two or more distinct fluorescent quinacridone derivatives, preferably having distinct fluorescent emission spectrums. In still further embodiments, oligonucleotides labeled with one or more fluorescent quinacridone derivatives comprise additional (e.g., fluorescent or non-fluorescent) labels.

In some embodiments, oligonucleotides further comprise fluorescent quenching groups. The present invention is not limited to a particular quenching group. Any quenching group that has an absorption spectrum that overlaps with the emission spectra of the fluorescent quinacridone derivative and is soluble and able to be attached to nucleic acids may be utilized in the present invention.

In preferred embodiments, nucleic acids are labeled with quinacridone derivatives by the attachment of the quinacridone to a phosphoramidite. In some preferred embodiments, the method described in Examples 4 and 5 is utilized. Such a method allows the incorporation of the phosphoramidite during nucleic acid synthesis at any position (e.g., 3′, 5′, or internal) of an oligonucleotide.

The present invention is not limited to the method described in Examples 4 and 5. Any method that results in the incorporation of a quinacridone into a nucleic acid may be utilized. For example, in some embodiments, linking molecules are used to attach quinacridones to nucleic acids. In some embodiments, oligonucleotides with 3′ substituent groups are generated using tri-functional linking groups (See e.g., U.S. Pat. No. 5,512,667, herein incorporated by reference). In other embodiments, additional linking groups are utilized including, but not limited to, those described in U.S. Pat. Nos. 5,212,304, 4,757,141, each of which is herein incorporated by reference.

In yet other embodiments, oligonucleotides are labeled at the 3′ end with quinacridones using a solid support comprising a quinacridone derivative attached via a linking group. Methods for the generation of solid supports suitable for the attachment of labels include, but are not limited to, those described in U.S. Pat. No. 5,736,626, 5,141,813, 6,015,895, each of which is herein incorporated by reference.

In still further embodiments, quinacridone derivatives are added following synthesis of the nucleic acid sequence of interest (See e.g., U.S. Pat. No. 6,194,563, herein incorporated by reference). In yet other embodiments, quinacridone derivatives are added to naturally-derived nucleic acids (e.g., genomic DNA) (See e.g., U.S. Pat. No. 5,491,224, herein incorporated by reference).

B. Uses of Quinacridone Nucleic Acid Conjugates

The quinacridone-nucleic acid conjugates of the present invention find use in a variety of applications. The nucleic acid conjugates of the present invention find use in any application that utilizes nucleic acid molecules comprising detectable labels.

In some embodiments, quinacridone dyes of the present invention are employed in fluorescence resonance energy transfer (FRET) based detection methods. In FRET, the fluorophore (e.g., quinacridone dye of the present invention) is quenched with a quencher moiety (e.g., on the same biological molecule or otherwise provided). The removal of the quencher results in de-quenching and detectable fluorescence. Examples of uses of quinacridone dyes in FRET reactions are provided in the experimental section below.

In other embodiments, quinacridone dyes may also act as labels through mechanisms other than FRET and simple quenching, for example, molecular beacons. In some embodiments, wavelength-shifting molecular beacons (See e.g., Tyagi et al., Nat. Biotechnol. 18: 1191-1196, 2000) are utilized. In some embodiments, molecular beacon containing nucleic acid probes are utilized that have three fluorescent dyes in relationship to one another based on the secondary structure of the nucleic acid. In the absence of target sequence, fluorescent donor emissions are quenched by a nearby quencher because of the hairpin structure of the probe. In the presence of target nucleic acid, hairpin unfolding releases the donor molecule, and a third nearby dye accepts the donor emission and emits energy at an altered wavelength due to FRET. This three-way relationship helps to distinguish between background fluorescence from the primary donor and bonafide signal generated from probe hybridization.

1. Nucleic Acid Detection

In some embodiments of the present invention, nucleic acid sequences labeled with quinacridone derivatives are used in the detection of nucleic acid sequences. For example, in some embodiments, labeled nucleic acid sequences are hybridized to target nucleic acid sequences in a hybridization assay. In a hybridization assay, the presence or absence of a target nucleic acid sequence is determined based on the ability of the nucleic acid from the sample to hybridize to a complementary nucleic acid molecule (e.g., a oligonucleotide probe labeled with a fluorescent quinacridone derivative of the present invention). A variety of hybridization assays using a variety of technologies for hybridization and detection are suitable for use in the detection of target nucleic acids. A description of a selection of assays is provided below.

a. Direct Detection of Hybridization

In some embodiments, hybridization of a nucleic acid sequence labeled with a quinacridone derivative of the present invention to the target sequence of interest is detected directly by visualizing a bound probe comprising a fluorescent quinacridone derivative of the present invention (e.g., a Northern or Southern assay; See e.g., Ausabel et al. (eds.), Current Protocols in Molecular Biology, John Wiley & Sons, NY [1991]). In a these assays, genomic DNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g., on an agarose gel) and transferred to a membrane. A nucleic acid sequence labeled with a quinacridone derivative of the present invention specific for the target nucleic acid sequence being detected is allowed to contact the membrane under conditions of low, medium, or high stringency. Unbound labeled nucleic acid is removed and the presence of binding is detected by visualizing the labeled nucleic acid.

b. Detection of Hybridization Using “DNA Chip” Assays

In some embodiments of the present invention, target sequences are detected using a DNA chip hybridization assay. In this assay, a series of nucleic acid probes are affixed to a solid support. Each of the probes is designed to be unique to a given target sequence. The DNA sample of interest is contacted with the DNA “chip” and hybridization is detected.

In some embodiments, the DNA chip assay is a GeneChip (Affymetrix, Santa Clara, Calif.; See e.g., U.S. Pat. Nos. 6,045,996; 5,925,525; and 5,858,659; each of which is herein incorporated by reference) assay. The GeneChip technology uses miniaturized, high-density arrays of oligonucleotide probes affixed to a “chip.” Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

In some embodiments, the nucleic acid to be analyzed is isolated, amplified by PCR, and labeled with a fluorescent quinacridone derivative of the present invention. The labeled DNA is then incubated with the array using a fluidics station. The array is then inserted into a scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

In other embodiments, a DNA microchip containing electronically captured probes (nucleic acid sequences labeled with a quinacridone derivative of the present invention) (Nanogen, San Diego, Calif.) is utilized (See e.g., U.S. Pat. Nos. 6,017,696; 6,068,818; and 6,051,380; each of which are herein incorporated by reference). Through the use of microelectronics, Nanogen's technology enables the active movement and concentration of charged molecules to and from designated test sites on its semiconductor microchip. DNA capture probes unique to a given SNP or mutation are electronically placed at, or “addressed” to, specific sites on the microchip. Since DNA has a strong negative charge, it can be electronically moved to an area of positive charge.

First, a test site or a row of test sites on the microchip is electronically activated with a positive charge. Next, a solution containing the DNA probes is introduced onto the microchip. The negatively charged probes rapidly move to the positively charged sites, where they concentrate and are chemically bound to a site on the microchip. The microchip is then washed and another solution of distinct DNA probes is added until the array of specifically bound DNA probes is complete.

A test sample is then analyzed for the presence of target DNA molecules by determining which of the DNA capture probes hybridize with complementary DNA in the test sample (e.g., a PCR amplified gene of interest). An electronic charge is also used to move and concentrate target molecules to one or more test sites on the microchip. The electronic concentration of sample DNA at each test site promotes rapid hybridization of sample DNA with complementary capture probes (hybridization may occur in minutes). To remove any unbound or nonspecifically bound DNA from each site, the polarity or charge of the site is reversed to negative, thereby forcing any unbound or nonspecifically bound DNA back into solution away from the capture probes. In some embodiments, a laser-based fluorescence scanner is then used to detect binding.

In still further embodiments, an array technology based upon the segregation of fluids on a flat surface (chip) by differences in surface tension (ProtoGene, Palo Alto, Calif.) is utilized (See e.g., U.S. Pat. Nos. 6,001,311; 5,985,551; and 5,474,796; each of which is herein incorporated by reference). Protogene's technology is based on the fact that fluids can be segregated on a flat surface by differences in surface tension that have been imparted by chemical coatings. Once so segregated, oligonucleotide probes are synthesized directly on the chip by ink-jet printing of reagents. The array with its reaction sites defined by surface tension is mounted on a X/Y translation stage under a set of four piezoelectric nozzles, one for each of the four standard DNA bases. The translation stage moves along each of the rows of the array and the appropriate reagent is delivered to each of the reaction site. For example, the A amidite is delivered only to the sites where amidite A is to be coupled during that synthesis step and so on. Common reagents and washes are delivered by flooding the entire surface and removing by spinning.

DNA probes unique for the target sequence of interest are affixed to the chip using Protogene's technology. The chip is then contacted with the PCR-amplified genes of interest. Following hybridization, unbound DNA is removed and hybridization is detected using any suitable method (e.g., by fluorescence de-quenching of an incorporated fluorescent quinacridone group).

In yet other embodiments, a “bead array” is used for the detection of polymorphisms (Illumina, San Diego, Calif.; See e.g., PCT Publications WO 99/67641 and WO 00/39587, each of which is herein incorporated by reference). Illumina uses a BEAD ARRAY technology that combines fiber optic bundles and beads that self-assemble into an array. Each fiber optic bundle contains thousands to millions of individual fibers depending on the diameter of the bundle. The beads are coated with an oligonucleotide specific for the detection of a given target sequence. Batches of beads are combined to form a pool specific to the array. To perform an assay, the BEAD ARRAY is contacted with a prepared subject sample (e.g., DNA). Hybridization is detected using any suitable method.

c. Enzymatic Detection of Hybridization

In some embodiments of the present invention, hybridization is detected by enzymatic cleavage of specific structures (e.g., the INVADER assay, Third Wave Technologies; See e.g., U.S. Pat. Nos. 5,846,717, 6,090,543; 6,001,567; 5,985,557; and 5,994,069; each of which is herein incorporated by reference). The INVADER assay detects specific DNA and RNA sequences by using structure-specific enzymes to cleave a complex formed by the hybridization of overlapping oligonucleotide probes. Elevated temperature and an excess of one of the probes enable multiple probes to be cleaved for each target sequence present without temperature cycling. These cleaved probes then direct cleavage of a second labeled probe. The secondary probe oligonucleotide can be 5′-end labeled with a fluorescent quinacridone derivative of the present invention that is quenched by an internal dye. Upon cleavage, the de-quenched quinacridone labeled product may be detected using a standard fluorescence plate reader.

The INVADER assay detects specific target sequences in unamplified genomic DNA. The isolated DNA sample is contacted with the first probe specific for the target sequence of interest and allowed to hybridize. Then a secondary probe, specific to the first probe, and containing the fluorescent quinacridone label, is hybridized and the enzyme is added. Binding is detected by using a fluorescent plate reader and comparing the signal of the test sample to known positive and negative controls.

In some embodiments, hybridization of a bound probe is detected using a TaqMan assay (PE Biosystems, Foster City, Calif.; See e.g., U.S. Pat. Nos. 5,962,233 and 5,538,848, each of which is herein incorporated by reference). The assay is performed during a PCR reaction. The TaqMan assay exploits the 5′-3′ exonuclease activity of the AMPLITAQ GOLD DNA polymerase. A probe, specific for a given target sequence, is included in the PCR reaction. The probe consists of an oligonucleotide with a 5′-reporter dye (e.g., a fluorescent quinacridone derivative) and a 3′-quencher dye. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In still further embodiments, polymorphisms are detected using the SNP-IT primer extension assay (Orchid Biosciences, Princeton, N.J.; See e.g., U.S. Pat. Nos. 5,952,174 and 5,919,626, each of which is herein incorporated by reference). In this assay, SNPs are identified by using a specially synthesized DNA primer and a DNA polymerase to selectively extend the DNA chain by one base at the suspected SNP location. DNA in the region of interest is amplified and denatured. Polymerase reactions are then performed using miniaturized systems called microfluidics. Detection is accomplished by adding a label (e.g., a fluorescent quinacridone derivative) to the nucleotide suspected of being at the target nucleic acid location. Incorporation of the label into the DNA can be detected by any suitable method (e.g., with a fluorimeter).

d. Other Detection Assays

The quinacridone derivatives of the present invention find use in additional detection assays including, but not limited to, enzyme mismatch cleavage methods (e.g., Variagenics, U.S. Pat. Nos. 6,110,684, 5,958,692, 5,851,770, herein incorporated by reference in their entireties); polymerase chain reaction; branched hybridization methods (e.g., Chiron, U.S. Pat. Nos. 5,849,481, 5,710,264, 5,124,246, and 5,624,802, herein incorporated by reference in their entireties); rolling circle replication (e.g., U.S. Pat. Nos. 6,210,884 and 6,183,960, herein incorporated by reference in their entireties); NASBA (e.g., U.S. Pat. No. 5,409,818, herein incorporated by reference in its entirety); molecular beacon technology (e.g., U.S. Pat. No. 6,150,097, herein incorporated by reference in its entirety); E-sensor technology (Motorola, U.S. Pat. Nos. 6,248,229, 6,221,583, 6,013,170, and 6,063,573, herein incorporated by reference in their entireties); cycling probe technology (e.g., U.S. Pat. Nos. 5,403,711, 5,011,769, and 5,660,988, herein incorporated by reference in their entireties); Dade Behring signal amplification methods (e.g., U.S. Pat. Nos. 6,121,001, 6,110,677, 5,914,230, 5,882,867, and 5,792,614, herein incorporated by reference in their entireties); ligase chain reaction (Barnay Proc. Natl. Acad. Sci USA 88, 189-93 (1991)); and sandwich hybridization methods (e.g., U.S. Pat. No. 5,288,609, herein incorporated by reference in its entirety).

In addition, the technologies available from a variety of commercial sources, including, but not limited to, Aclara BioSciences, Haywood, Calif.; Agilent Technologies, Inc., Palo Alto, Calif.; Aviva Biosciences Corp., San Diego, Calif.; Caliper Technologies Corp., Palo Alto, Calif.; Celera, Rockville, Md.; CuraGen Corp., New Haven, Conn.; Hyseq Inc., Sunnyvale, Calif.; Incyte Genomics, Palo Alto, Calif.; Applera Corp., Foster City, Calif.; Rosetta Inpharmatics, Kirkland, Wash.; and Sequenom, San Diego, Calif. are amenable to the incorporation of the quinacridone derivatives of the present invention.

2. Nucleic Acid Sequencing

In some embodiments, quinacridone labeled nucleic acids are utilized in nucleic acid sequencing (e.g., automated sequencing) methods (See e.g., U.S. Pat. Nos. 5,171,534, 5,374,527, and 4,855,225; each of which is herein incorporated by reference in its entirety). In some embodiments, a set of four quinacridones with different fluorescent emission spectra are utilized. Each of the quinacridones is coupled chemically to a primer that is used to initiate the synthesis of nucleic acid fragments. In turn, each tagged primer is then paired with one dideoxynucleotide and used in a primed synthesis reaction with a DNA polymerase. In other embodiments, the four quinacridones are attached to the C₇ position of a purine terminating base and the C₅ of a pyrimidine terminating base (See e.g., Prober et al. Science, 238:336 [1987]). In either embodiments, a fluorescence detector can then be used to detect the fluorophore-labeled DNA fragments. The four different dideoxy-terminated samples can be run in the same lane. Base sequence is then determined, for example, by analyzing the fluorescent signals emitted by the fragments as they pass a stationary detector during the separation process.

3. In vivo and In situ Applications

In some embodiments, the present invention provides in vivo and in situ methods that utilizing quinacridone labeled nucleic acids. Such methods find use in the analysis of nucleic acids in cells and populations of cells in culture.

a. FACS

In some embodiments, quinacridone derivatives are used to label or “stain” populations of cells so that each cell can be identified and quantitated based upon its fluorescence signal. In some embodiments, quinacridones are attached to nucleic acids that bind to cell surfaces. A computer collects the fluorescence signature of each cell and displays the pattern of fluorescence for the user to analyze. In other applications, where one might want to separate cells which have a certain staining pattern from all other cells (e.g., due to binding to a labeled pre-selected antigen), the flow cytometry machine can direct those desired cells into a tube provided by the user. This is called fluorescence activated cell sorting (FACS).

b. FISH

In some embodiments, quinacridone labeled nucleic acids are used in FISH (Fluorescence In-Situ Hybridization) procedures. A FISH sample is prepared by using multiple probes, each of which binds to a different DNA sequence in the chromosomes in the sample. Each probe is labeled with a different quinacridone dye (e.g., with different colors of emission spectra) or combination of two or more dyes.

III. Quinacridone Labeling of Additional Biological Molecules

The present invention is not limited to the labeling of nucleic acids with quinacridone derivatives. In some embodiments, additional biological molecules, including but not limited to, proteins (e.g., antibodies, peptides, and polypeptides), lipids, and carbohydrates are labeled with quinacridone derivatives.

A. Methods of Labeling

In some embodiments, the present invention provides proteins labeled with quinacridone derivatives of the present invention. In some embodiments, quinacridones are attached to a protein at a site that is remote from the active site of the protein by the use of exopeptidase and a nucleophile which is an amino acid, amino acid derivative, amine or alcohol (See e.g., U.S. Pat. No. 5,234,820, herein incorporated by reference). In other embodiments, conventional nucleophilic reaction conditions are utilized (See e.g., U.S. Pat. No. 6,224,644, herein incorporated by reference). In still further embodiments, proteins are labeled using methods described in Pramanik et al., Biochemistry 2001:10839 [2001] and U.S. Pat. No. 6,225,050, herein incorporated by reference.

In other embodiments, the present invention provides carbohydrates (e.g., saccharides) labeled with quinacridone derivatives of the present invention. Any suitable method may be utilized, including but not limited to, the method disclosed in U.S. Pat. No. 6,207,163, herein incorporated by reference.

B. Uses of Labeled Biological Molecules

Quinacridone labeled biological molecules find use in a variety of diagnostic and analytical methods. In some embodiments, quinacridone labeled molecules are utilized in in vivo imaging techniques. For example, in some embodiments, quinacridone derivatives of the present invention are utilized in methods of fluorescently imaging the carbohydrate uptake activity in living tissues (See e.g., U.S. Pat. Nos. 6,207,136 and 5,408,996; each of which is herein incorporated by reference). Such methods are useful, for example, in localizing malignant tissue and determining changes in viability of living tissue.

In some embodiments, labeled proteins are used in FACS methods (see above description). In other embodiments, antibodies are labeled with quinacridone dyes of the present invention. Such antibodies are useful in a variety of diagnostic methods involving the detection of antigens. The present invention is not limited to the methods disclosed herein. Any method utilizing labeled biological macromolecules is contemplated by the present invention.

IV. Other Uses

The present invention is not limited to the use of quinacridone dyes as biological molecule labels. In some further embodiments, the quinacridone dyes of the present invention are utilized may also be used as passive reference standards, as opposed to as directly conjugated labels.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); l or L (liters); ml (milliliters); μl (microliters); cm (centimeters); mm (millimeters); μm (micrometers); nm (nanometers); min (minutes); F.W. (formula weight); and ° C. (degrees Centigrade).

### Example 1

### Derivatization of Magenta and Magenta B Quinacridones

This example describes the derivatization of quinacridones (Magenta and Magenta B) with Br—(CH₂)₃—O-DMT using the synthesis procedure described in U.S. Pat. No. 5,725,651. The derivatization was performed to convert the starting material into the fluorescent derivative and to increase the solubility of the starting quinacridone. The results of this experiment indicated that the described method is not suitable for the derivatization of quinacridones with Br—(CH₂)₃—O-DMT.

The derivatization is an alkylation reaction performed in organic solvents such as tetrahyrofuran, N,N-dimethylformamide, dioxane, DMSO, N,N-dimethylacetamide or N-methylpyrrolidone and in the presence of sodium hydride as a strong base:

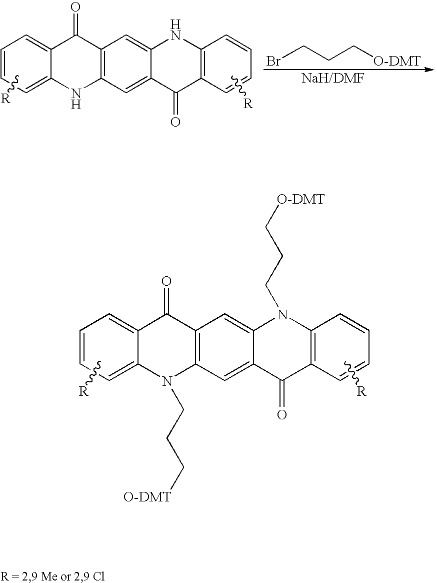

The alkylation of the 2,9-chloroquinacridone and 2,9-dimethylquinacridone (Magenta B and Magenta respectively) using DMT protected 3-Bromo propanol-1 was performed according to the protocol described in the patent.

0.0001847 mol of the pigment (R═Me—Magenta; R═Cl—Magenta B from Sun Chemical) was suspended in 5 ml of dry DMF in 25 ml round bottom reaction flask. The suspension was protected from moisture and stirred magnetically under a blanket of argon. 30 mg of a 60% NaH suspension in oil (Aldrich; 0.001133 mol of NaH) was added. The reaction was stirred under argon at room temperature for 48 hours. A dark blue color developed.

0.001133 mol (0.5 g) of DMT protected 3-bromo 1-propanol was dissolved in 1 ml of dry DMF and added to the reaction mixture and the mixture was stirred at room temp. Subsequently, 10 mg of tetrabutyl ammonium iodide was added to the stirred reaction mixture. After 4 hrs TLC analysis did not reveal the formation of the new reaction product.

Subsequently the reaction mixture was stirred and heated to 80° C. for 5 min and then 50 mg of the 60% NaH emulsion was added and the reaction mixture was heated again for an additional 5 min at 80° C.

After 50 min of stirring at room temperature, a 0.5 g (0.001133 mol) portion of DMT protected 3-bromo 1-propanol dissolved in 1.5 ml of the DMF was added. Then reaction mixture was heated to 80° C. for 15 min and then stirred at room temperature for 15 min. Subsequently, more of the 60% emulsion of the sodium hydride (100 mg) was added, the reaction mixture was heated to 80° C. and stirred for 15 min. TLC analysis indicated the formation of a very non-uniform reaction mixture without the formation of a dominant product.

### Example 2

### Derivatization of Magenta and Magenta B Quinacridones

In this Example, an alternate synthetic protocol was applied. This protocol utilizes Phase Transfer Catalysis in which a heterogeneous mixture of a saturated solution of sodium hydroxide and the inert organic solvent is used as a reaction medium. This method avoids the use of sodium hydride, which represents a dangerous material. In a number of small-scale experiments a new efficient protocol for the derivatization of quinacridones was developed.

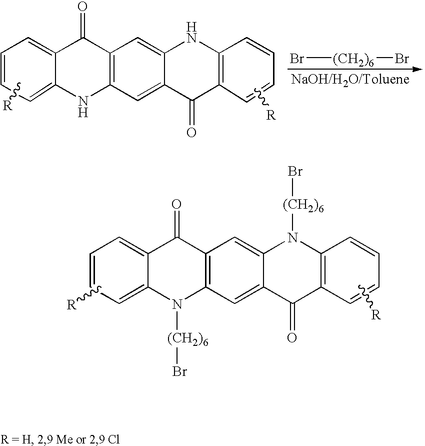

Alkylation experiments were performed using commercially available 1,6-dibromohexane.

### A. Synthesis of the dibromo-derivative of 2,9-dichloroquinacridone

1 g (0.006247 mol) of 2,9-chloroquinacridone (Magenta B, F.W. 381) was suspended in 50 ml of saturated NaOH/ Water: 50 ml toluene and magnetically stirred. 1.125 g (0.00305 mol) of tetrabutyl ammonium iodide was added and subsequently the resulting mixture was stirred at 50° C. for 15 min and at room temp for 50 min. A dark-blue color was developed.

Subsequently, 14.7 ml (22.7 g, 0.09306 mol) of 1,6-dibromohexane was added and the resulting reaction mixture was heated to the reflux for 15 min. The heat source was then removed and the reaction mixture was stirred and allowed to cool for 30 min. The resulting mixture was poured into the separatory funnel containing 50 ml toluene and 100 ml of a saturated NaCl/water solution. The organic layer was separated and washed with a solution of 100 ml of water and 35 ml of acetic acid. The organic layer was separated again and washed with a solution of 15 g of NaCl in 100 ml of water. Finally, the separated organic layer was dried over magnesium sulfate for 2 hrs and concentrated under reduced pressure (water aspirator).

Subsequently, 100 ml of hexane was added to the semi-liquid residue. The precipitated solid material was filtered off and washed with hexane (3×10 ml) and air-dried. The yield of the semi-solid material was 2.448 g (theoretical yield 1.85 g), which indicates the presence of organic solvents or moisture. The same derivative synthesized according to this protocol was synthesized earlier on the smaller scale (yield 0.108 g) and purified by column chromatography (silica 70-230 mesh; dichloromethane/3% methanol).

TLC (Merck silica plates) of the purified material was next performed. The mobil phase was dichloromethane/2.5% methanol; R_{f}=0.78. Mass Spectral analysis of the purified material confirmed its structure (F.W. 707).

### B. Synthesis of Bis-hydroxyl derivative of 2,9-dichloriquinacridone

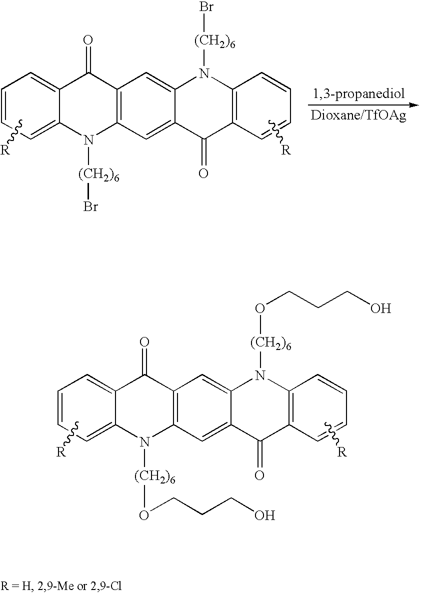

2.438 g (0.003448 mol) of the dibromo-derivative of 2,9-dichloroquinacridone synthesized in Section A above was dissolved in a solution of 10 ml of anhydrous 1,3-propanediol (Aldrich) and 20 ml of dry Dioxane (Aldrich). The resulting solution was stirred until a homogenous solution was formed (˜45 min).

Subsequently, 5 g (0.01945 mol) of Silver Trifluoromethanesulfonate (Aldrich, F.W. 256.94) was added. The resulting reaction mixture was initially stirred for 15 min at room temperature and subsequently heated to reflux for 15 min. Finally, the mixture was stirred for 30 min to allow the mixture to cool.

The resulting reaction mixture was poured into 300 ml of a saturated solution of sodium chloride/water and 100 ml of dichloromethane. The organic layer was separated and the water layer was additionally extracted with dichloromethane (4×50 ml). The combined extracts were dried over magnesium sulfate for 4 hours then concentrated under reduced pressure.

The product was isolated by column chromatography on silica 70-230 mesh, dichloromethane/10% methanol—20% methanol. A yield of 1.2 g (50%; theoretical 2.40 g) was obtained. TLC (Merck silica plates) of the purified material (mobil phase—dichloromethande/10% methanol) gave R_{f}=0.25. Mass Spectral analysis supported the desired structure (theoretical F.W. 697).

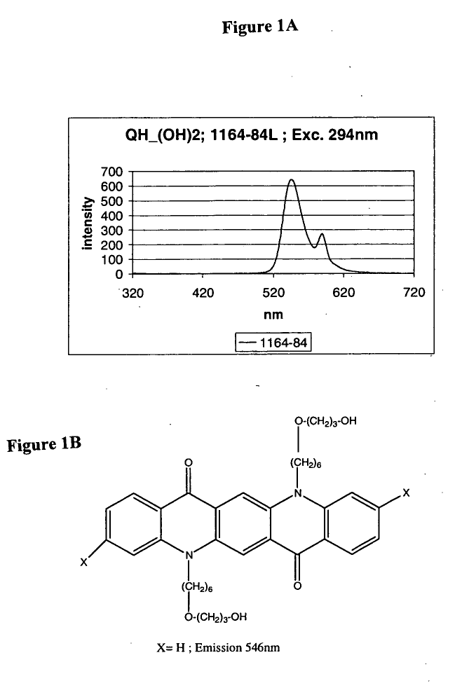

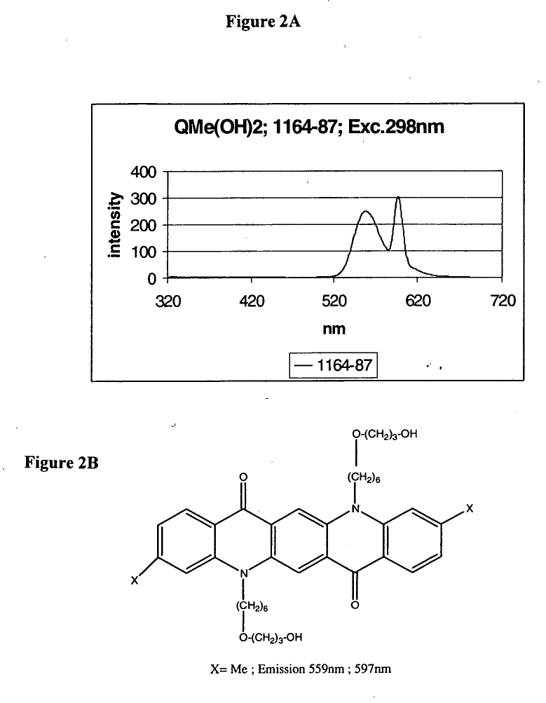

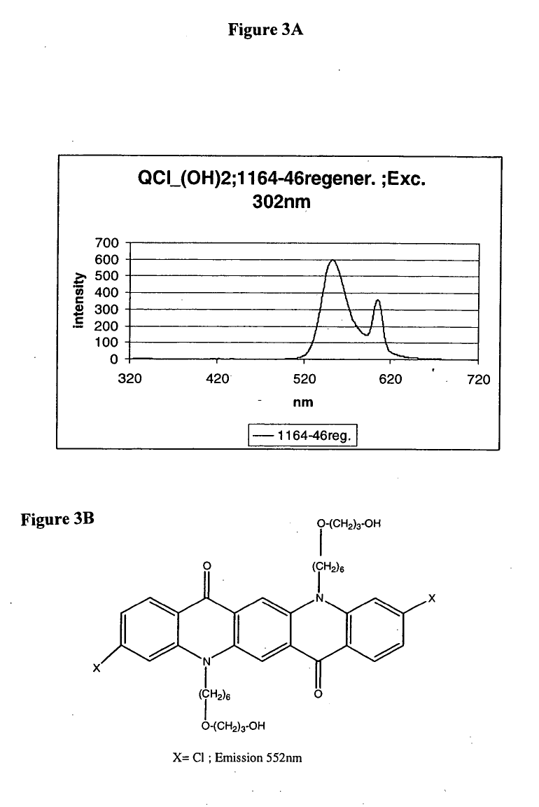

According to the above-described protocol three quinacridone derivatives were synthesized. Methanol solutions of those materials showed strong fluorescence when excited at the appropriate wavelengths (FIGS. 1, 2, and 3).

### Example 3

### DMT protection of the bis-hydroxyl derivative of 2,9-dichloroquinacridone

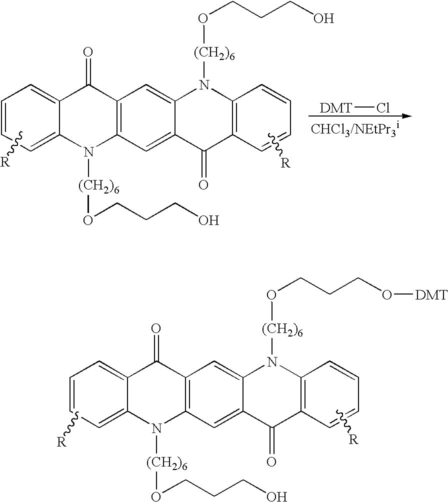

This example describes the DMT protection of the bis-hydroxyl derivative of 2,9-dichloroquinacridone in order to facilitate the attachment of the quinacridone to a phosphoramidite. 0.5344 g (0.0007667 mol) of the bis-hydroxyl derivative synthesized in Example 2B (F.W. 697) was dissolved in a solution of 4 ml of dry chloroform (Aldrich) and 0.5 ml (0.371 g, 0.00287 mol) of ethyl triisopropylamine (Aldrich). Subsequently, 0.1 g (0.0002951 mol) of dimethoxytrityl chloride (Aldrich) was added and the resulting reaction mixture was stirred overnight under dry nitrogen.

TLC analysis (Merck silica plates, mobil phase—dichloromethane/10% Methanol) indicated the formation of new material; R_{f}=0.6. After concentration under reduced pressure, the residue was re-dissolved in a minimal volume of dichloromethane/10% methanol and applied to a silica column (70-230 mesh/dichloromethane/10% methanol). Product containing fractions were combined and concentrated; yield of the isolated material was 0.088 g (30%; theoretical yield: 0.295 g). Mass Spectral analysis confirmed the structure of the material (F.W. 999).

### Example 4

### Synthesis of the phosphoramidite of DMT-protected 2,9-dichloroquinacridone

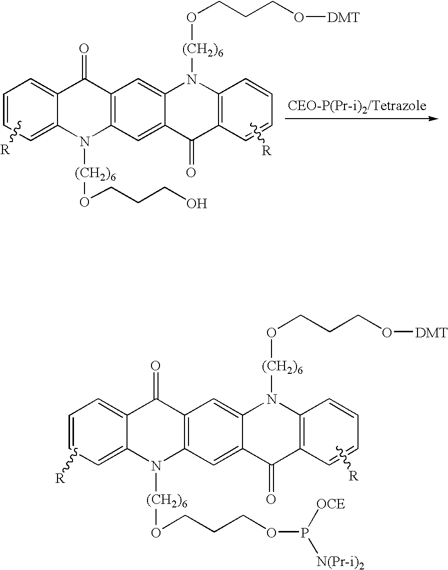

A solution of the mono-DMT protected quinacridone derivative synthesized in Example 3 containing 187.3 μMol (0.1841 g) of the material in 5.5 ml of anhydrous THF was prepared. The reaction mixture was protected from moisture and stirred magnetically at room temp. In the next step, 0.07 ml (0.0002204 mol) of 2-cyanoethyl tetraisopropylphosphoramidite (Aldrich, F.W. 301.42; d 0.949) was added to the stirred solution of the quinacridone derivative. Subsequently, a solution of 15 mg (0.000214 mol) of tetrazole in 1.5 ml of acetonitrile was added to the resulting reaction mixture. Stirring was continued at room temperature for 70 min.

TLC analysis (Merck silica plates, mobil phase—dichloromethane/5% methanol/5% triethylamine) indicated that the reaction was completed (product R_{f}=0.94). The reaction mixture was poured into 50 ml of 5% NaHCO₃/1 ml triethylamine/20 ml dichloromethane. The organic layer was separated and the water layer was extracted additionally with dichloromethane 2×10 ml. The organic solutions were combined and dried over magnesium sulfate for 30 min. After filtration, the resulting solution was concentrated under reduced pressure and the residue was co-evaporated with 5 ml of acetonitrile. The residue was dried over phosphorus pentoxide under high vacuum. The final yield was 0.2237 g (95%; theoretical yield—0.2329 g).

### Example 5

### Modification of DNA with bischloroquinacridone phosphoramidite

A solution of 0.2237 g (0.0001771 mol) of the quinacridone phosphoramidite in 3 mL of anhydrous THF (59 μMol/ml) was prepared. One μmol of CPG containing the desired sequence was transferred into a 2.5 ml gas tight Hamilton syringe. The CPG solid support was washed with dichloromethane (2×1 ml) and subsequently treated with 5 ml of a 3% solution of dichloroacetic acid in dichloromethane for 1 min (DMT deprotection)
Subsequently, the following steps were performed:
Wash with 1:1 Acetonitryle/Pyridine 3×1 ml
Wash with Acetonitryle 6×1 ml
Wash with THF 3×1 mL

The quinacridone phosphoramidite was next coupled to the DNA probe as follows. lmL of the THF solution of the quinacridone phosphoramidite (59 μMol/mL) was drawn into the syringe. Subsequently, 0.6 mL of the tetrazole solution in Acetonitrile (10 mg/0.6 mL) was taken into the syringe. Contents of the syringe were agitated. After a coupling time of 15 min, the solution was expelled from the syringe as follows:
Wash Acetonitryle 6×1 mL
Wash THF 3×1 mL

The coupling step was then repeated with the same amount of the reagents. The coupling time was 25 min. The solution was then expelled from the syringe and washed as follows:
Wash 1:1 Acetonitrile/Py 6×1 mL
Wash Acetonitrile 3×1 mL
Wash THF 3×1 mL

The oxidation step was next performed as follows: 1 mL of the standard oxidizing solution (12/THF/Py; ABI reagent for automated DNA synthesis) was drawn into the syringe; reaction time—3 min; Wash 1:1 Acetonitryle/Py 6×1 mL; Wash acetonitrile 3×1 mL; Wash DCM 6×1 mL.

The detritylation step was next performed as follows: DMT deprotection was performed with 5 mL of 3% solution of dichloroacetic acid in dichloromethane for a reaction time of 1 min with an estimated coupling yield—32%, followed by:
Wash 1:1 acetonitrile/Py 3×1 mL
Wash acetonitrile 6×1 mL
Wash DCM 6×1 mL

The solid support was dried under reduced pressure and then treated with ammonia for 12 hrs at 55° C. Product containing ammonia solution was concentrated. The quinacridone labeled DNA material was isolated by Reverse Phase HPLC (semipreparative Dionex C18 column, 10×250 mm , flow 2 ml/min, mobil phase 0.1M TEAA/Acetonitrile, gradient 1% acetonitrile per min). The product-containing fraction was concentrated under reduced pressure and desalted on a NAP column using standard desalting protocol.

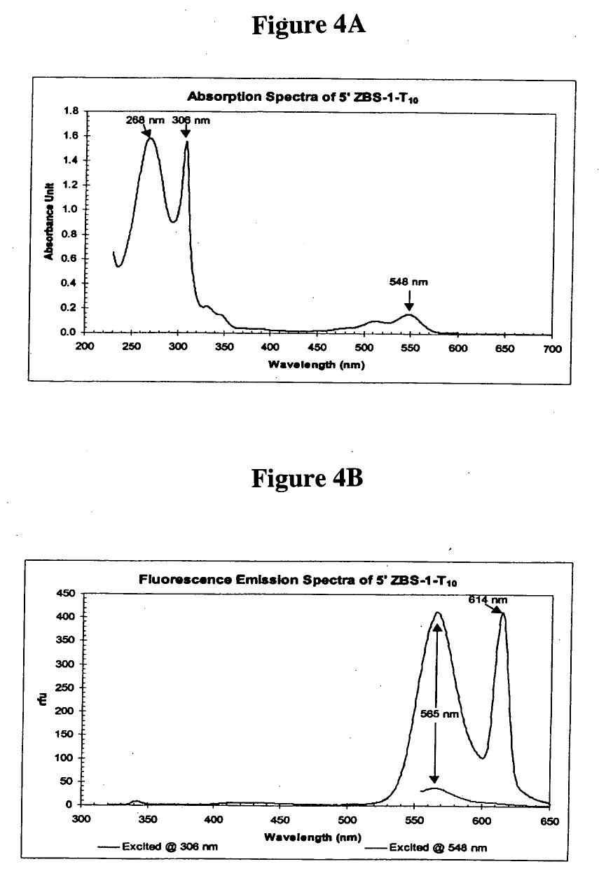

In a first experiment, a conjugate of a dT10-mer (SEQ ID NO: 1) labeled at its 5′-end with the bischloroquinacridone derivative was synthesized. The absorption and fluorescence spectra were measured (FIG. 4). Strong fluorescence was observed when the solution of the 5′-labeled dT10 oligonucleotide was excited at 306 nm wavelength but much weaker when excited at 546 nm wavelength.

In a second experiment, a conjugate of a second oligonucleotide (SEQ ID NO: 2) labeled at its 5′-end with the bischloroquinacridone derivative was synthesized.

### Example 6

### INVADER Assay Turnover Experiment

Two oligonucleotides, (SEQ ID NOs: 1 and 2), labeled with the quinacridone dye prepared in Examples 2-5 above at the 5′ end were synthesized, gel purified and tested for probe turnover rate under standard INVADER assay conditions. The TET-labeled probe 594-41-4 was used as a control to measure relative turnover rates.

The INVADER assay was performed with 2 μM of 594-61-1, 594-61-2 or 594-41-4 probes, 0.5 μM INVADER oligonucleotide 594-38-5 (SEQ ID NOs: 1 and 2), 1 nM target oligonucleotide 594-41-6 (SEQ ID NO: 3), and 10 ng/μl AfuFEN1 CLEAVASE enzyme or 10 ng/μl AveFEN1 CLEAVASE enzyme in a 10 μL solution of 10 mM MOPS, pH 7.5, 4 mM MgCl₂, 20 ng/μl tRNA (Sigma), 0.05% Tween 20 and 0.05% NP40 at 63° C. for 8 min. Control experiments were performed under the same conditions in the absence of the target oligonucleotide.

The samples were assembled on ice, overlaid with Chill-out liquid wax (MJ Research) and transferred to a Mastercycler heating block (Eppendorf). The reactions were terminated by the addition of 10 μL of 95% formamide containing 20 mM EDTA and 0.02% methyl violet. One microliter aliquots of each reaction were loaded on a 100×100×1 mm slab of 15% denaturing polyacrylamide gel (crosslinked 19:1) with 7 M urea in a buffer containing 45 mM Tris borate, pH 8.3 and 1 mM EDTA. An electric field of 12 watts power was applied for 15 minutes. The intensities of bands corresponding to the products and uncleaved probes were measured using a FMBIO-100 fluorescence imager (Hitachi, Alameda, Calif.) equipped with 532-nm laser and 585-nm filter at 40% sensitivity level. The turnover rate for each Probe was determined as described (Lyamichev, Biochemistry 2000). The results are shown in Table 1. The results indicate that both of the quinacridone containing oligonucleotides are able to function as probes in the INVADER assay.

TABLE 1
INVADER Assay Data

| Probe | Enzyme | Turnover, I/min | % of 594-41-4 rate |
|---|---|---|---|
| 594-61-1 | AfuFEN1 | 25 | 38 |
| 594-61-1 | AveFEN1 | 55 | 60 |
| 594-61-2 | AveFEN1 | 32 | 48 |
| 594-61-2 | AfuFEN1 | 72 | 79 |
| 594-41-4 | AfuFEN1 | 68 | |
| 594-41-4 | AveFEN1 | 92 | |

### Example 7

### INVADER Squared Assay

This Example evaluates the performance of the ZBS-2 quinacridone dye in a FRET reaction. The quinacridone dye is utilized in the secondary oligonucleotide of the INVADER assay.

A. Methods

Two FRET oligos were synthesized with the ZB2 dye in the 5′ position. One of the oligos has Dabcyl as a quencher in position 4, the other has Z28 as a quencher in position 4. Both FRET oligos are complementary to arm sequence 1. All reactions were carried out in 96 well ultra-generic plates (containing CleavaseVIII and buffers, but no FRET oligos), and read on the Safire plate reader.
The structure of the quinacridone phosphoramidite used to introduce the quinacridone modification named ZB2: 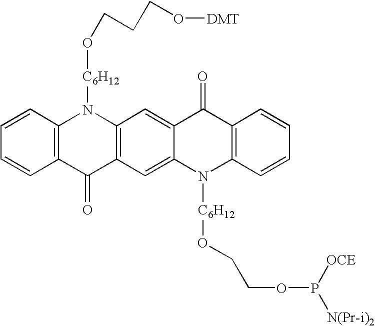
The structure of the Dabcyl phosphoramidite (Glen Research) used as a quencher in the construction of the FRET cassette; Cat. # 10-1058-xx

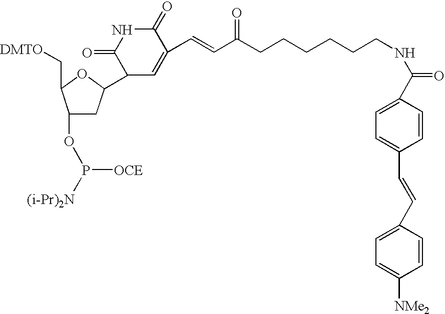

Z28 (Glen Research): 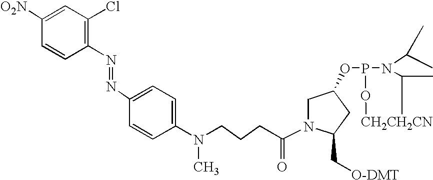
A. Results

1. Monoplex and Triplex Reactions of ZB2-Dabcyl, Red Dye and FAM.

Monoplex and triplex reactions of ZB2, FAM, and Red dye on soybean genomic DNA, using soybean alcohol dehydrogenase (adh), β-tubulin-2 (tub), and Cauliflower Mosaic Virus 35S promoter (CaMV) probe sets. In the monoplex reactions all reporter dyes were reporting for tub with arm 1. In the triplex reaction ZB2-Dabcyl was reporting tub with arm 1, FAM was reporting adh with arm 2, and Red dye was reporting CaMV with arm 3. Reactions were carried out for 6 hours at 63° C. on 35 ng soybean genomic DNA (0.05 attomoles).

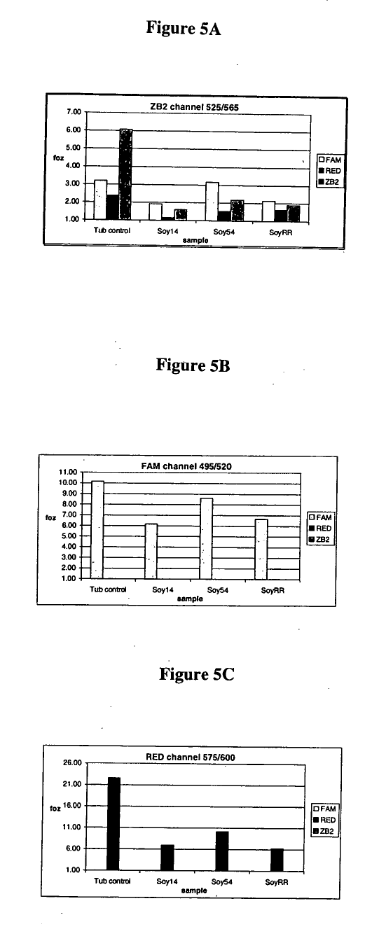

Monoplex results (See FIG. 5 A-C) show that ZB2 reporter dye does not cross-talk in FAM or RED channel. When using 525/565 in a monoplex fashion, both RED and FAM show cross-talk. Moving the excitation of ZB2 to 546 nm eliminates FAM, but not RED, cross-talk.

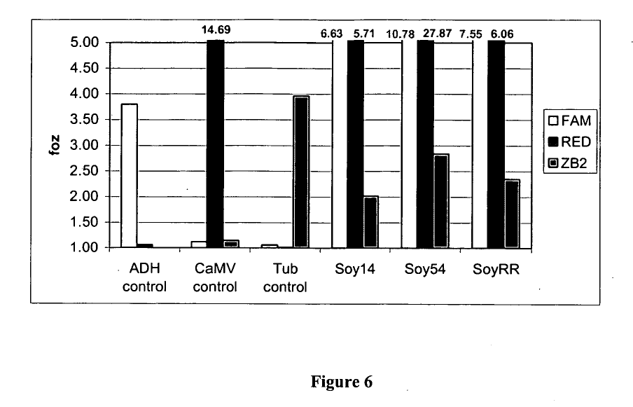

The results of the triplex experiment are shown in FIG. 6. Triplex scanning conditions were as follows: ZB2 525 nm/565 nm, FAM 495 nm/520 nm, RED dye 575 nm/600 nm. ZB2 bandwidths were 2.5nm, FAM and RED bandwidths were 5 nm. The absence of the ZBS-2 signal in the synthetic controls for ADH and CaMV indicate that in the triplex format there is no cross-talk from FAM or RED in the ZB2 channel.

Other conditions that were evaluated in this experiment were different excitation and emission wavelengths for ZB2. At excitation 306 nm and emission 565 nm, or 614 nm, both RED dye and FAM showed significant cross-talk in both mono-and triplex reactions. As excitation 546 nm emission 565 nm, or 614 nm, there was no FAM cross-talk but significant RED dye cross-talk in both mono- and triplex reactions. The results show that excitation 525 nm, emission 565 nm (bandwidth 2.5 nm) are the optimal conditions for a triplex reaction.

2. Comparison between ZB2-Dabcyl and ZB2-Z28 FRET Oligos, Monoplex and Biplexed with FAM

In this experiment the ZB2-Dabcyl probe is compared to the ZB2-Z28 probe in monoplex format, as well as in biplex format with FAM. Also a FAM/RED biplex is included for comparison between RED and both ZB2 FRET oligos. The assay used is the Porcine Stress Syndrome (PSS) assay. The wild-type C-probe has arm 1 and is reported by ZB2-Dabcy1, ZB2-Z28 or Red dye, the mutant T-probe has arm 2 and is reported by FAM. Reactions ere carried out for 4 hours at 65° C. on 100 ng of denatured porcine DNA (0.05 attomoles).

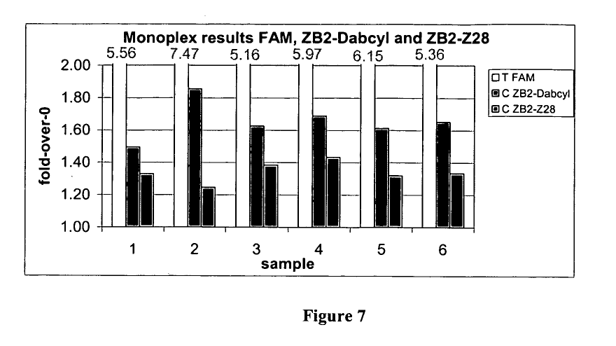

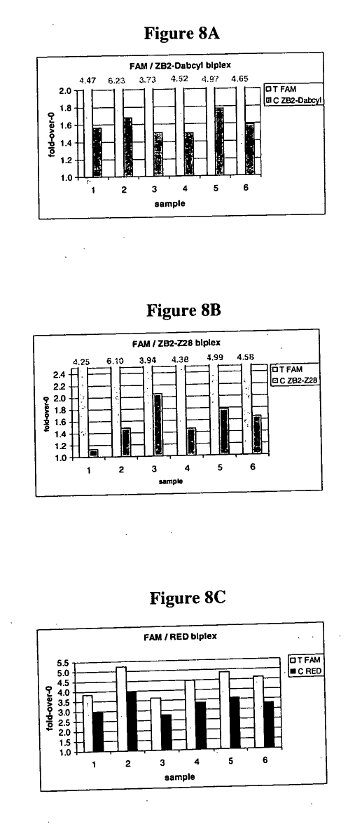

FIG. 7 shows monoplex reactions of the PSS assay, using FAM and ZB2 reporter dyes. Excitation/emission wavelengths are: FAM 495nm/520 nm, ZB2 dyes 546 nm/565 nm. FIG. 8 shows biplex reactions of ZB2—and RED dye with FAM. Excitation emission wavelengths are: FAM 495 nm/520 nm, ZB2 546 nm/565 nm, RED dye 575 nm/600 nm. The results indicate that ZB2-Dabcy1 is more robust in signal generation than ZB2-Z28. The average performance of ZB2-Dabcy1 compared to RED dye is 26%.

In conclusion, in a triplex system there seems to be no cross-talk between channels if the following settings are chosen:

| | Excitation | Emission | bandwidth |
|---|---|---|---|
| ZB2 | 525 nm | 565 nm | 2.5 nm |
| FAM | 495 nm | 520 nm | 5.0 nm |
| RED | 575 nm | 600 nm | 5.0 nm |

In a monoplex system, cross-talk of FAM and RED in ZB2 channel does occur, while ZB2 shows no cross-talk in FAM or RED channels. Excitation at 546 nm and emission at 565 nm for ZB2 shows significant improvement of signal to noise ratio over excitation 525 nm emission 565 nm. However, these settings cause significant cross-talk of red dye in both monoplex and triplex formats.

### Example 8

### Evaluation of ZB2 with Different Linkers

This Example describes the evaluation of the performance of different linkers in the INVADER DNA Assay.

The INVADER assay for PSS was setup with the following FRET oligos:

FAM
RED
# 1273-38 and # 1273-78:
5′-dye-tct-Quencher-t-cgg-cct-ttt-ggc-cga-gag-acc-
tcg-gcg-cg-Hexanediol-3
# 1273-62:
3 × C3 linker-5′-dye-tct-Quencher-t-cgg-cct-ttt-ggc-
cga-gag-acc-tcg-gcg-cg-Hexanediol-3
# 1273-72:
5′-dye-tct-Quencher-18 atom linker-t-cgg-cct-ttt-
ggc-cga-gag-acc-tcg-gcg-cg-Hexanediol-3
# 1273-74:
5′-dye-tct-Quencher-C3 linker-t-cgg-cct-ttt-ggc-
cga-gag-acc-tcg-gcg-cg-Hexanediol-3
1273-76:
5′-dye-tct-Quencher-3 × C3 linker-t-cgg-cct-ttt-
ggc-cga-gag-acc-tcg-gcg-cg-Hexanediol-3

Reactions were run in monoplex for 4 hours at 63° C. on 200 ng of heterozygous DNA.

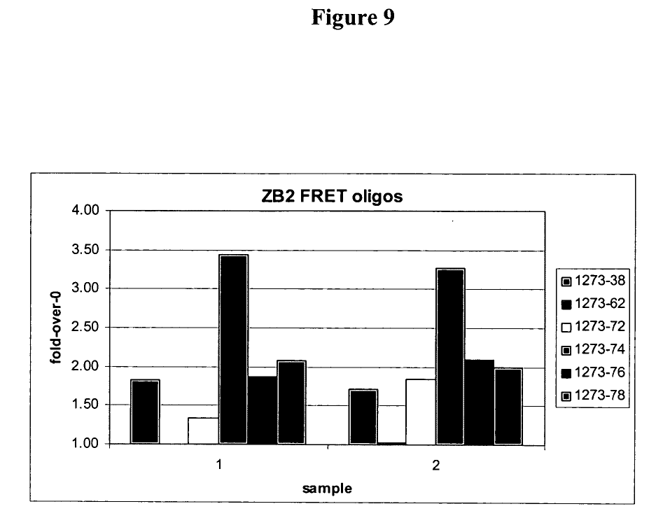

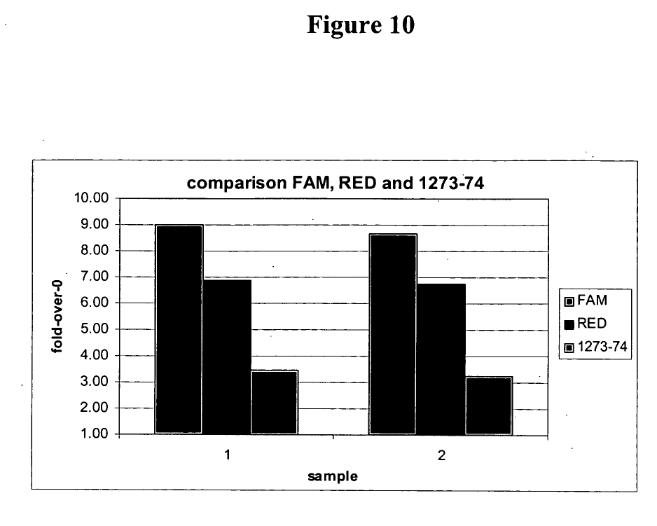

FIG. 9 shows the results of reactions using just the ZB2 FRET oligos. The FRET oligo 1273-74 gives a boost in signal generation. Oligos 1273-38 and 1273-78 use the same linker, but are two different syntheses. They have comparable performance. FIG. 10 illustrates a comparison between 1273-74 and the FAM and RED FRET oligos. Both FAM and RED have higher performance than the 1273-74 ZB2 FRET oligo.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A composition comprising a biological molecule, said biological molecule comprising a quinacridone.

2. The composition of claim 1, wherein said biological molecule is a nucleic acid.

3. The composition of claim 2, wherein said quinacridone has the structure:

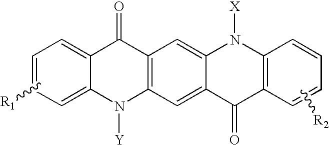

4. The composition of claim 3, wherein X is (CH₂)₆—O—(CH₂)₃—OH and Y is (CH₂)₆—O—(CH₂)₃—OH.

5. The composition of claim 3, wherein X and Y are independently selected from the group consisting of hydrogen, halogen, amide, hydroxyl, cyano, nitro, azido, mono- or di-nitro-substituted benzyl, amino, mono- or di-C₁-C₄ alkyl-substituted amino, sulphydryl, carbonyl, carboxyl, C₁-C₆ alkoxy, acrylate, vinyl, styryl, aryl, heteroaryl, C₁-C₂₀ alkyl, aralkyl, sulphonate, sulphonic acid, quaternary ammonium, E-F and (CH₂)ₙ-G, wherein E is a spacer group having a chain from 1-60 atoms selected from the group consisting of carbon, nitrogen, oxygen, sulphur and phosphorus atoms and F is a target bonding group, and G is selected from the group consisting of sulphonate, sulphate, phosphonates, phosphate, quaternary ammonium and carboxyl and n is an integer from 1 to 6.

6. The composition of claim 3, wherein R₁ and R₂ are independently selected from the group consisting of hydrogen, halogen, amide, hydroxyl, cyano, nitro, azido, mono- or di-nitro-substituted benzyl, amino, mono- or di-C₁-C₄ alkyl-substituted amino, sulphydryl, carbonyl, carboxyl, C₁-C₆ alkoxy, acrylate, vinyl, styryl, aryl, heteroaryl, C₁-C₂₀ alkyl, aralkyl, sulphonate, sulphonic acid, quaternary ammonium, E-F and (CH₂)ₙ-G, wherein E is a spacer group having a chain from 1-60 atoms selected from the group consisting of carbon, nitrogen, oxygen, sulphur and phosphorus atoms and F is a target bonding group, and G is selected from the group consisting of sulphonate, sulphate, phosphonates, phosphate, quaternary ammonium and carboxyl and n is an integer from 1 to 6.

7. The composition of claim 2, wherein said nucleic acid is selected from the group consisting of ssDNA, ssRNA, dsDNA, dsRNA, and PNA.

8. The composition of claim 2, wherein said quinacridone is covalently linked to said nucleic acid.

9. The composition of claim 2, wherein said nucleic acid is an oligonucleotide.

10. The composition of claim 9, wherein said oligonucleotide further comprises a fluorescence quenching molecule.

11. A kit comprising a biological molecule, said biological molecule comprising a quinacridone.

12. The kit of claim 11, wherein said quinacridone has the structure:

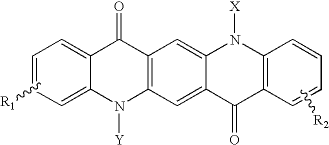

13. The kit of claim 11, wherein said biological molecule is a nucleic acid.

14. The kit of claim 11, further comprising a second nucleic acid, wherein said second nucleic acid comprises a fluorescent molecule with a different fluorescence emission spectrum than said quinacridone.

15. The kit of claim 11, wherein said kit further comprises reagents for performing a detection assay selected from the group consisting of an INVADER assay, a TAQMAN assay, a SNP-IT assay, a Southern blot, and an array assay.

16. A method of detecting a target nucleic acid sequence, comprising,
a) providing
i) a nucleic acid comprising a quinacridone;
ii) a sample comprising target nucleic acid; and
b) contacting said sample with said nucleic acid under conditions such that said target nucleic acid sequence is detected.

17. The method of claim 16, wherein said quinacridone has the structure:

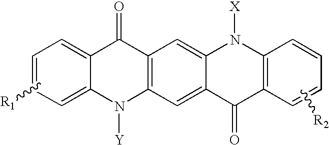

18. The method of claim 17, wherein X is (CH₂)₆—O—(CH₂)₃—OH and Y is (CH₂)₆—O—(CH₂)₃—OH.

19. The method of claim 16, wherein said contacting comprises a nucleic acid detection assay.

20. The method of claim 16, wherein said detection assay is selected from the group consisting of INVADER assay, TAQMAN assay, SNP-IT assay, Southern blot, and an array assay.
